(19) United States
(12) Patent Application Publication
(10) Pub. No.: US 2010/0297734 A1
(43) Pub. Date: Nov. 25, 2010

(54) Fusion Tag Comprising an Affinity Tag and an EF-Hand Motif Containing Polypeptide and Methods of Use Thereof

(75) Inventors: Andrew J. McCluskey, Toronto (CA); Gregory M.K. Poon, Pullman, WA (US); Jean Gariépy, Toronto (CA)

Correspondence Address: Ballard Spahr LLP, SUITE 1000, 999 PEACHTREE STREET, ATLANTA, GA, 30309-3915, US

(73) Assignee: University Health Network, Toronto, ON (CA)

(21) Appl. No.: 12/682,718

(22) PCT Filed: Oct. 10, 2008
(86) PCT No.: PCT/CA08/01773

§ 371 Date: Aug. 6, 2010

Related U.S. Application Data
(60) Provisional application No. 60/979,670, filed on Oct. 12, 2007.

(51) Int. Cl. C12N 1/00; C12N 15/00; C12N 15/63; C07K 14/00; C07K 1/22

(52) U.S. Cl. 435/243; 536/23.1; 435/320.1; 530/350; 530/344

(57) ABSTRACT

The application discloses a fusion tag comprising an affinity tag and a polypeptide comprising one or more EF hand motif(s). Preferably, said fusion tag comprises a polyhistidine tag, one or more EF hand motif(s) of calmodulin and a thrombin cleavage site. Methods of using said fusion tag to purify a polypeptide of interest are also disclosed.

### FIELD OF THE INVENTION

The present application relates to improved protein purification methods and materials. In particular, the application discloses a novel fusion tag comprising an affinity tag and a polypeptide comprising one or more EF hand motif(s), and methods of use thereof.

### BACKGROUND OF THE INVENTION

A quarter of all new pharmaceuticals entering the marketplace are recombinant therapeutic proteins (Pavlou and Reichert 2004; Walsh 2005). A recurrent challenge in manufacturing such proteins has been the development of rapid, robust and economical protein purification methods. A similar requirement for homogeneous preparations exists for proteins intended for structural solution by X-ray crystallography and NMR spectroscopy. Proteomic approaches linked to mass spectrometry require a universal purification approach for the recovery of distinct, low-abundance proteins from complex yeast and mammalian cell lysates (Rigaut et al. 1999; Puig et al. 2001). The purification of recombinant proteins has been simplified by affinity chromatographic strategies in which a peptide or protein affinity tag, cloned in frame with the target construct, selectively interacts with a ligand which has been immobilized on a solid support. The most common affinity tag is a polyhistidine tag, typically (His)₆, which selectively binds to transition metal ions such as Ni²⁺ and Co²⁺ in a procedure known as immobilized metal-affinity chromatography (IMAC). Although IMAC is routinely used for the purification of recombinant proteins, purity under native conditions is often suboptimal due to nonspecific binding and co-elution of host (particularly eukaryotic) proteins with His-tagged targets (Lichty et al. 2005).

In addition to IMAC, other purification tags have been developed including natural or designed peptides as well as protein modules such as maltose binding protein (MBP), protein A (ProtA) and glutathione S-transferase (GST) (Stevens 2000). As in the case of IMAC, none of these purification tags alone is sufficient in most instances to purify recombinant proteins to homogeneity starting from bacterial or eukaryotic cell extracts. This challenge has led to an array of dual (and multiple) affinity tags in order to improve protein expression (Sachdev and Chirgwin 1998) or product purity (Lichty et al. 2005). These complex tags typically combine the (His)₆ peptide with other affinity tags or epitopes such as calmodulin binding peptide (CBP) (Honey et al. 2001), GST (Panagiotidis and Silverstein 1995; Coulombe and Meloche 2002), MBP (Kapust and Waugh 1999; Donnelly et al. 2006; Tropea et al. 2007), thioredoxin (Yeliseev et al. 2007), Strep tags (Mueller et al. 2003; Yeliseev et al. 2007), hemagglutinin (Winkler et al. 1998; Honey et al. 2001), the HIV-Tat peptide (Goda et al. 2007), and the FLAG epitope (Pathak and Imperiali 1997). These approaches vary significantly in terms of product purity, yield, purification conditions, procedural complexity, and cost. An ideal multiple affinity purification procedure should exhibit the following features: (i) substantial gain in purity, (ii) small tag, (iii) complete removal of the affinity tag after purification, (iv) rapid and robust procedures (requiring few or no buffer exchanges between steps), (v) compatibility with native conditions, and (vi) low cost.

### SUMMARY OF THE INVENTION

Disclosed herein is a novel fusion tag comprising an affinity tag and a polypeptide comprising one or more EF hand motif(s), and methods of using this novel fusion tag to purify polypeptides. The affinity tag is optionally a polyhistidine tag, such as (His)₆. The polypeptide comprising one or more EF hand motif(s) is optionally calmodulin.

Accordingly, one aspect relates to a novel fusion tag, which can be used to purify recombinant proteins. The fusion tag comprises an affinity tag attached to a polypeptide comprising one or more EF hand motif(s). In one embodiment, the fusion tag comprises a polyhistidine tag attached to calmodulin.

Another aspect disclosed herein relates to an isolated nucleic acid sequence comprising a nucleic acid sequence that encodes a fusion tag, wherein the fusion tag comprises an affinity tag attached to a polypeptide comprising one or more EF hand motif(s).

Another aspect is an expression vector that comprises an isolated nucleic acid sequence disclosed herein.

A further aspect is a host cell that comprises an expression vector disclosed herein.

An additional aspect is a method of purifying a polypeptide using the nucleic acid sequences, expression vectors and/or host cells disclosed herein. For example, the polypeptide can be expressed using an expression vector that comprises a nucleic acid sequence that encodes the fusion tag attached to the polypeptide, and then the polypeptide can be purified using at least two different purification steps or methods. In an embodiment, a cleavage recognition site is situated between the fusion tag and the polypeptide and the fusion tag can be cleaved from the polypeptide at the cleavage recognition site.

An additional aspect includes kits comprising the isolated nucleic acid sequences, the expression vectors, and/or host cells described herein and an ancillary agent and/or instructions for use.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

Embodiments of the invention will now be described in relation to the drawings in which:

FIG. 1 shows the construction of the HiCaM purification tag. The HiCaM-tag combines a (His)₆ sequence for IMAC purification followed by CaM for Hydrophobic Interaction Chromatography (HIC) purification with phenyl sepharose. The terminal NcoI/NdeI restriction sites (underlined) allow the DNA sequence coding for the tag to be inserted directly into a pET15b vector 5′ to the multiple cloning site. The (His)₆ tag and thrombin cleavage sequences are marked.

FIG. 2 depicts the IMAC/HIC purification of HiCaM-tagged proteins. (A) The N-terminal HiCaM purification tag combines a (His)₆ sequence for IMAC purification followed by a CaM module for HIC purification with phenyl sepharose. By virtue of the thrombin cleavage site (TCS), the target protein can either be cleaved on-column or eluted and subsequently cleaved with thrombin. (B) Aliquots containing 3 μg of total protein corresponding to each purification step were resolved by SDS-PAGE followed by Coomassie Blue staining. HiCaM-tagged proteins were obtained by elution from phenyl sepharose (Lanes 7 and 10) with 50 mM TrisHCl, 1 mM EGTA, pH 7.5 and (for tandem IMAC/HIC purified constructs) subsequently cleaved with thrombin (Lane 11) to release the HiCaM tag (light arrow) from the target protein (dark arrow). Untagged constructs were also directly obtained by on-column thrombin cleavage (Lane 12). (C) Histogram highlighting the purity of proteins (eGFP, solid bars; human p53(1-360); open bars) recovered from IMAC purification step alone, HIC step alone, HiCaM tandem steps, and following the removal of the HiCaM purification tag (derived from densitometry measurements performed on lanes 4, 7, 10 and 12, from FIG. 2b).

FIG. 3 shows functional assays of tandem IMAC/HIC-purified constructs following HiCaM tag removal. (A) Fluorescence excitation and emission spectra (excited at 488 nm) of purified eGFP (3 μM) were recorded from 300 to 700 nm at 25° C. (B) Human p53(1-360) bound to a specific ³²P-labelled DNA recognition sequence (5′-AGGCATGTCTAGGCATGTCT-3′) as monitored by electrophoretic mobility shift (McLure and Lee 1998). (C) SDS-PAGE of the p53 construct following chemical crosslinking with glutaraldehyde displays a band ladder characteristic of tetramer formation via its oligomerization domain (residues 325 to 355) (Poon et al. 2007).

FIG. 4 depicts a representative SDS-PAGE analysis of TAP-eGFP purified by IgG and CaM affinity chromatography. Purity for the cleared lysate (Lane 1) as well as after IgG (Lane 4) and CaM affinity chromatography (Lane 7) was determined densitometrically and shown in Table 2.

FIG. 5 shows the purification of phenyl sepharose-bound

HiCaM fusion constructs displaying the C-terminal seven amino acids (lanes 3 and 4, and 12 and13), 11 amino acids (lanes 5 and 6, and 14 and 15), 17 amino acids (lanes 7 and 8, and 16 and 17) of P1 and P2, or HiCaM alone (lanes 1 and 2, and 10 and 11). Fusion constructs were incubated briefly with the SLT-1 A1 chain (lane 9) or ricin A chain (lane 18) and separated by SDS PAGE followed by staining with Coomassie brilliant blue. The black arrow indicates the purified A1 chain, and the gray arrow represents the HiCaM tag seen in all lanes. FT, column flow-through (unbound A1 chain and HiCaM); TC, thrombin-cleaved peptide.

### DETAILED DESCRIPTION OF THE INVENTION

Disclosed herein is a novel fusion tag comprising an affinity tag, such as a polyhistidine tag (optionally (His)₆), and a polypeptide comprising one or more EF hand motif(s) (optionally calmodulin) and methods of using this novel fusion tag to purify recombinant proteins.

One aspect of the present application is a novel fusion tag, which can be used to purify recombinant proteins. The fusion tag comprises an affinity tag attached to a polypeptide comprising one or more EF hand motif(s).

The term “affinity tag” as used herein refers to an amino acid sequence that is used to facilitate purification of a protein or polypeptide. In one embodiment, the affinity tag includes a streptavidin tag, a c-myc tag, an HA-tag, a T7 tag, a FLAG-tag, a polyhistidine tag (such as (His)₆), a polyarginine tag, a polyphenylalanine tag, a polycysteine tag, or a polyaspartic acid tag. In a specific embodiment, the affinity tag is (His)₆. The term “(His)₆” as used herein refers to the following amino acid sequence: HHHHHH.

The term “polypeptide comprising one or more EF hand motif(s)” as used herein refers to a polypeptide which is 5-50, 5-25, 5-20, 5-15 or 5-10 kDa and comprises at least one EF hand motif, and has hydrophobic matrix binding activity and is suitable for hydrophobic interaction chromatography. A person skilled in the art will appreciate that there are a number of polypeptides that comprise one or more EF hand motif(s), which are suitable for the methods disclosed herein. In one embodiment, the polypeptide comprising one or more EF hand motif(s) includes but is not limited to trophonin C, S-100, parvalbumin, oncomodulin or calmodulin. In one specific embodiment, the polypeptide comprising one or more EF hand motif(s) is calmodulin.

The term “calmodulin” as used herein refers to a small calcium-binding protein found in nature in eukaryotic cells. The term includes calmodulin, irrespective of source. For example, calmodulin is optionally obtained from a wild type eukaryotic source, from a recombinant prokaryotic or eukaryotic source or from other synthetic sources. In one embodiment, calmodulin has following amino acid sequence (SEQ ID NO:1):

1 madqlteeqi aefkeafslf dkdgdgtitt kelgtvmrsl gqnpteaelq dminevdadd

61 pgngtidfp efltmmarkm kdtdseeeir eafrvfdkdg ngyisaaelr hvmtnlgekl

121 tdeevdemir eadidgdgqv nyeefvqmmt ak

The term “EF hand motif' as used herein refers to a metal ion-binding motif that consists of a helix-loop-helix structure. In a specific embodiment, the polypeptide comprising one or more EF hand motif(s) comprises one or more EF hand motifs of calmodulin. Calmodulin has 4 EF hand motifs, which are defined as residues 20 to 31, 56 to 67, 93 to 104 and 129 to 140 of SEQ ID NO:1.

The term “amino acid” includes all of the naturally occurring amino acids as well as modified amino acids.

In one embodiment of the fusion tag, the affinity tag is attached directly to the polypeptide comprising one or more EF hand motif(s). In another embodiment, the affinity tag is attached via a fusion linker to the polypeptide comprising one or more EF hand motif(s). A person skilled in the art will appreciate that the fusion linker can be any amino acid sequence or length, provided that the fusion linker does not interact adversely with the structure of the polypeptide to be purified or the purification of the polypeptide. The fusion linker is preferably 1 to 100, 1 to 75, 1 to 50, 1 to 25, 1 to 10, 1 to 5 or 1 to 3 amino acids in length. In a specific embodiment, the fusion linker is 3 amino acid sequences. In a more specific embodiment, the fusion linker has the amino acid sequence: SSG.

In a further embodiment, the linker can comprise a cleavage recognition site so that the affinity tag can be cleaved from the polypeptide comprising one or more EF hand motif(s). The term “cleavage recognition site” as used herein includes any amino acid sequence that is recognized by a sequence specific protease, and includes a tev, thrombin, enterokinase, factor Xa, furin, or geninase I cleavage recognition site.

A person skilled in the art will appreciate that the affinity tag portion of the fusion tag can be located C-terminus or N-terminus to the polypeptide comprising one or more EF hand motif(s) portion of the fusion tag.

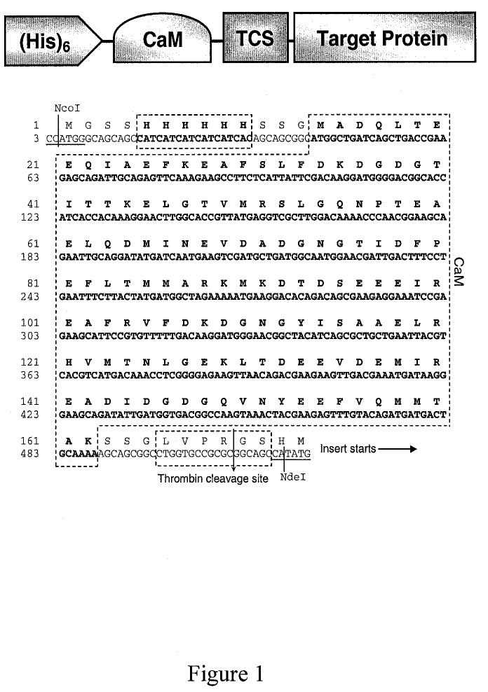

In one embodiment, the fusion tag comprises all or part of the amino acid sequence shown in SEQ ID NO:2 (FIG. 1).

Another aspect of the present application is an isolated nucleic acid sequence comprising a nucleic acid sequence that encodes the fusion tag disclosed herein. In one embodiment, the isolated nucleic acid sequence comprises all or part of the nucleic acid sequence shown in SEQ ID NO:3 (FIG. 1).

The term “nucleic acid sequence” as used herein refers to a sequence of nucleoside or nucleotide monomers consisting of naturally occurring bases, sugars and intersugar (backbone) linkages. The term also includes modified or substituted sequences comprising non-naturally occurring monomers or portions thereof. The nucleic acid sequences of the present application may be deoxyribonucleic acid sequences (DNA) or ribonucleic acid sequences (RNA) and may include naturally occurring bases including adenine, guanine, cytosine, thymidine and uracil. The sequences may also contain modified bases. Examples of such modified bases include aza and deaza adenine, guanine, cytosine, thymidine and uracil; and xanthine and hypoxanthine. The term “nucleic acid” is intended to include DNA and RNA and can be either double stranded or single stranded, and represents the sense or antisense strand.

The term “isolated nucleic acid sequences” or “isolated protein” as used herein refers to a nucleic acid or protein substantially free of cellular material or culture medium when produced by recombinant techniques.

The novel fusion tag can be used to purify polypeptides produced using recombinant techniques. Accordingly, in one embodiment, the isolated nucleic acid sequence comprising the nucleic acid sequence that encodes the fusion tag disclosed herein further comprises a nucleic acid sequence that encodes a polypeptide. A person skilled in the art will appreciate that the isolated nucleic acid sequence can be designed so that upon expression, the polypeptide is attached directly to the fusion tag. In another embodiment, the polypeptide is attached via a linker to the fusion tag. A person skilled in the art will appreciate that the fusion linker can be any amino acid sequence or length, provided that the fusion linker does not interact adversely with the structure of the polypeptide to be purified or the purification of the polypeptide. In one embodiment the linker is 1 to 100, 1 to 75, 1 to 50, 1 to 25, 1 to 10, 1 to 5 or 1 to 3 amino acids in length. In a further embodiment, the linker can comprise a cleavage recognition site so that the polypeptide can be cleaved from the fusion tag. In a specific embodiment, the cleavage recognition site is a thrombin cleavage recognition site. A person skilled in the art will appreciate that the fusion tag can be C-terminus or N-terminus to the polypeptide.

A person skilled in the art will appreciate the polypeptide can be positioned between the affinity tag and polypeptide comprising one or more EF hand motif(s) portion of the fusion tag, and can be flanked on either or both sides by a linker, which optionally comprises a cleavage recognition site.

In another embodiment, the isolated nucleic acid sequence comprising a nucleic acid sequence that encodes the fusion tag disclosed herein further encodes a cleavage recognition site. In one embodiment, the isolated nucleic acid sequence is designed so that upon expression the cleavage recognition site is attached directly to the fusion tag. In another embodiment, the cleavage recognition site is attached via a linker to the fusion tag. A person skilled in the art will appreciate that the linker can be any amino acid sequence or length, provided that the linker does not interact adversely with the structure of the polypeptide to be purified or the purification of the polypeptide. In one embodiment the linker is 1 to 100, 1 to 75, 1 to 50, 1 to 25, 1 to 10, 1 to 5 or 1 to 3 amino acids in length. In a specific embodiment, the linker is 3 amino acid sequences. In a more specific embodiment, the linker has the amino acid sequence: SSG. A person skilled in the art will appreciate that the cleavage recognition site can be C-terminus or N-terminus to the fusion tag.

The present application includes variants of the specific amino acid and nucleotide sequences disclosed herein. The term “variant” as used herein includes modifications or chemical equivalents of the amino acid and nucleotide sequences of the present invention that perform substantially the same function as the proteins or nucleic acid molecules of the invention in substantially the same way.

In one embodiment, variants of proteins of the invention include, without limitation, conservative amino acid substitutions. Variants of proteins of the invention also include additions and deletions to the proteins disclosed herein. In addition, variant peptides and variant nucleotide sequences include analogs and derivatives thereof.

For example, a variant of SEQ ID NO:1, which is the amino acid sequence of calmodulin, will still be a small, water-soluble protein and have the function of being able to bind to hydrophobic matrices (e.g. HIC). For instance, a variant of calmodulin includes fragments thereof that have hydrophobic matrix binding activity and are suitable for hydrophobic interaction chromatography. In one embodiment, the fragment includes one or more of the EF hand motifs of calmodulin (i.e. one or more of the amino acid sequences defined by 20 to 31, 56 to 67, 93 to 104 and 129 to 140 of SEQ ID NO:1).

In another example, a variant of SEQ ID NO:2, which is the amino acid sequence of a specific embodiment of the novel fusion tag, will still have (His)₆, calmodulin and a cleavage recognition site in tandem and have the function of being able to bind to hydrophobic matrices (e.g. HIC) and IMAC matrices, and the cleavage recognition site will allow cleavage of the fusion tag from the polypeptide.

A “conservative amino acid substitution”, as used herein, is one in which one amino acid residue is replaced with another amino acid residue without abolishing the peptide's desired properties.

The term “derivative of a peptide” refers to a peptide having one or more residues chemically derivatized by reaction of a functional side group. Such derivatized molecules include for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Also included as derivatives are those peptides which contain one or more naturally occurring amino acid derivatives of the twenty standard amino acids. For examples: 4-hydroxyproline may be substituted for proline; 5-hydroxylysine may be substituted for lysine; 3-methylhistidine may be substituted for histidine; homoserine may be substituted for serine; and ornithine may be substituted for lysine.

In one embodiment, the term variant includes nucleic acid or amino acid sequences that have 80%, 90%, 95%, 98% or 99% to the specific nucleic acid or amino acid sequences disclosed herein.

To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions.times.100%). In one embodiment, the two sequences are the same length.

The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules of the present invention. BLAST protein searches can be performed with the XBLAST program parameters set, e.g., to score-50, wordlength=3 to obtain amino acid sequences homologous to a protein molecule of the present invention. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., the NCBI website). Another preferred, non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

A person skilled in the art will appreciate that the novel nucleic acid sequences of the present application can be used in a number of recombinant methods. In particular, the novel fusion tag can be used to facilitate the purification of polypeptides produced using recombinant techniques as shown in Example 1. The novel fusion tag can also be used for isolating polypeptides for pull-down experiments as shown in Example 2.

Accordingly, the nucleic acid sequences of the present application may be incorporated in a known manner into an appropriate expression vector which ensures good expression of the proteins encoded thereof. Possible expression vectors include but are not limited to cosmids, plasmids, or modified viruses (e.g. replication defective retroviruses, adenoviruses and adeno-associated viruses), so long as the vector is compatible with the host cell used. The expression vectors are “suitable for transformation of a host cell”, which means that the expression vectors contain a nucleic acid molecule of the present application and regulatory sequences selected on the basis of the host cells to be used for expression, which is operatively linked to the nucleic acid molecule. Operatively linked is intended to mean that the nucleic acid is linked to regulatory sequences in a manner which allows expression of the nucleic acid.

The present application therefore contemplates a recombinant expression vector of the present application containing a nucleic acid molecule of the present application, or a fragment thereof, and the necessary regulatory sequences for the transcription and translation of the inserted protein-sequence.

Suitable regulatory sequences may be derived from a variety of sources, including bacterial, fungal, viral, mammalian, or insect genes (For example, see the regulatory sequences described in Goeddel, Gene Expression Technology: Methods in Enzymology 185, Academic Press, San Diego, Calif. (1990)). Selection of appropriate regulatory sequences is dependent on the host cell chosen as discussed below, and may be readily accomplished by one of ordinary skill in the art. Examples of such regulatory sequences include: a transcriptional promoter and enhancer or RNA polymerase binding sequence, a ribosomal binding sequence, including a translation initiation signal. Additionally, depending on the host cell chosen and the vector employed, other sequences, such as an origin of replication, additional DNA restriction sites, enhancers, and sequences conferring inducibility of transcription may be incorporated into the expression vector.

The recombinant expression vectors of the present application may also contain a selectable marker gene which facilitates the selection of host cells transformed or transfected with a recombinant molecule of the present application. Examples of selectable marker genes are genes encoding a protein such as G418 and hygromycin which confer resistance to certain drugs, β-galactosidase, chloramphenicol acetyltransferase, firefly luciferase, or an immunoglobulin or portion thereof such as the Fc portion of an immunoglobulin preferably IgG. Transcription of the selectable marker gene is monitored by changes in the concentration of the selectable marker protein such as β-galactosidase, chloramphenicol acetyltransferase, or firefly luciferase. If the selectable marker gene encodes a protein conferring antibiotic resistance such as neomycin resistance transformant cells can be selected with G418. Cells that have incorporated the selectable marker gene will survive, while the other cells die. This makes it possible to visualize and assay for expression of recombinant expression vectors of the present application and in particular to determine the effect of a mutation on expression and phenotype. It will be appreciated that selectable markers can be introduced on a separate vector from the nucleic acid of interest.

The recombinant expression vectors may also contain genes which encode a fusion moiety which provides increased expression of the recombinant protein; increased solubility of the recombinant protein; and aid in the purification of the target recombinant protein by acting as a ligand in affinity purification. For example, a proteolytic cleavage site may be added to the target recombinant protein to allow separation of the recombinant protein from the fusion moiety subsequent to purification of the fusion protein. Typical fusion expression vectors include pGEX (Amrad Corp., Melbourne, Australia), pMal (New England Biolabs, Beverly, Mass.) and pRIT5 (Pharmacia, Piscataway, N.J.) which fuse glutathione S-transferase (GST), maltose E binding protein, or protein A, respectively, to the recombinant protein.

Recombinant expression vectors can be introduced into host cells to produce a transformed host cell. The terms “transformed with”, “transfected with”, “transformation” and “transfection” are intended to encompass introduction of nucleic acid (e.g. a vector) into a cell by one of many possible techniques known in the art. The term “transformed host cell” as used herein is intended to also include cells capable of glycosylation that have been transformed with a recombinant expression vector of the present application. Prokaryotic cells can be transformed with nucleic acid by, for example, electroporation or calcium-chloride mediated transformation. For example, nucleic acid can be introduced into mammalian cells via conventional techniques such as calcium phosphate or calcium chloride co-precipitation, DEAE-dextran mediated transfection, lipofectin, electroporation or microinjection. Suitable methods for transforming and transfecting host cells can be found in Sambrook et al. (Molecular Cloning: A Laboratory Manual, 3rd Edition, Cold Spring Harbor Laboratory Press, 2001), and other laboratory textbooks.

Suitable host cells include a wide variety of eukaryotic host cells and prokaryotic cells. For example, the proteins of the present application may be expressed in yeast cells or mammalian cells. Other suitable host cells can be found in Goeddel, Gene Expression Technology: Methods in Enzymology 185, Academic Press, San Diego, Calif. (1991). In addition, the proteins of the present application may be expressed in prokaryotic cells, such as Escherichia coli (Zhang et al., Science 303(5656): 371-3 (2004)). In addition, a Pseudomonas based expression system such as Pseudomonas fluorescens can be used (US Patent Application Publication No. US 2005/0186666, Schneider, Jane C et al.).

Yeast and fungi host cells suitable for carrying out the present application include, but are not limited to Saccharomyces cerevisiae, the genera Pichia or Kluyveromyces and various species of the genus Aspergillus. Examples of vectors for expression in yeast S. cerevisiae include pYepSec1 (Baldari. et al., Embo J. 6:229-234 (1987)), pMFa (Kurjan and Herskowitz, Cell 30:933-943 (1982)), pJRY88 (Schultz et al., Gene 54:113-123 (1987)), and pYES2 (Invitrogen Corporation, San Diego, Calif.). Protocols for the transformation of yeast and fungi are well known to those of ordinary skill in the art (see Hinnen et al., Proc. Natl. Acad. Sci. USA 75:1929 (1978); Itoh et al., J. Bacteriology 153:163 (1983), and Cullen et al. (BiolTechnology 5:369 (1987)).

Mammalian cells suitable for carrying out the present application include, among others: COS (e.g., ATCC No. CRL 1650 or 1651), BHK (e.g. ATCC No. CRL 6281), CHO (ATCC No. CCL 61), HeLa (e.g., ATCC No. CCL 2), 293 (ATCC No. 1573) and NS-1 cells. Suitable expression vectors for directing expression in mammalian cells generally include a promoter (e.g., derived from viral material such as polyoma, Adenovirus 2, cytomegalovirus and Simian Virus 40), as well as other transcriptional and translational control sequences. Examples of mammalian expression vectors include pCDM8 (Seed, B., Nature 329:840 (1987)) and pMT2PC (Kaufman et al., EMBO J. 6:187-195 (1987)).

Given the teachings provided herein, promoters, terminators, and methods for introducing expression vectors of an appropriate type into plant, avian, and insect cells may also be readily accomplished. For example, within one embodiment, the proteins of the present application may be expressed from plant cells (see Sinkar et al., J. Biosci (Bangalore) 11:47-58 (1987), which reviews the use of Agrobacterium rhizogenes vectors; see also Zambryski et al., Genetic Engineering, Principles and Methods, Hollaender and Setlow (eds.), Vol. VI, pp. 253-278, Plenum Press, New York (1984), which describes the use of expression vectors for plant cells, including, among others, PAPS2022, PAPS2023, and PAPS2034).

Insect cells suitable for carrying out the present application include cells and cell lines from Bombyx, Trichoplusia or Spodotera species. Baculovirus vectors available for expression of proteins in cultured insect cells (SF 9 cells) include the pAc series (Smith et al., Mol. Cell Biol. 3:2156-2165 (1983)) and the pVL series (Lucklow, V. A., and Summers, M. D., Virology 170:31-39 (1989)). Some baculovirus-insect cell expression systems suitable for expression of the recombinant proteins of the present application are described in PCT/US/02442.

Alternatively, the proteins of the present application may also be expressed in non-human transgenic animals such as rats, rabbits, sheep and pigs (Hammer et al. Nature 315:680-683 (1985); Palmiter et al. Science 222:809-814 (1983); Brinster et al. Proc. Natl. Acad. Sci. USA 82:4438-4442 (1985); Palmiter and Brinster Cell 41:343-345 (1985) and U.S. Pat. No. 4,736,866).

Accordingly, the present application provides a recombinant expression vector comprising one or more of the novel nucleic acid sequences disclosed herein as well as methods and uses of the expression vectors in the preparation of recombinant proteins. Further, the application provides a host cell comprising one or more of the novel nucleic acid sequences or expression vectors comprising one or more of the novel nucleic acid sequences. In one embodiment, the host cell is E. coli.

An additional aspect of the invention is a method of purifying a polypeptide produced using recombinant techniques using the nucleic acid sequences, expression vectors and/or host cells disclosed herein. For example, the polypeptide can be expressed using an expression vector that comprises a nucleic acid sequence that encodes the fusion tag attached to the polypeptide, and then the polypeptide can be purified via the fusion tag using two different purification steps or methods. For instance, one purification step is a purification step specific for the affinity tag portion of the fusion tag and the second purification step is a purification step specific for the polypeptide comprising one or more EF hand motif(s) portion of the fusion tag. The purification steps or methods can be done in any order. A person skilled in the art will appreciate that the purification step or method will depend on what affinity tag is used. For example, if the affinity tag is a polyhistidine tag, then immobilized metal affinity chromatography can be used.

In a specific embodiment, the fusion tag comprises (His)₆ and calmodulin, and the polypeptide is purified using immobilized metal-affinity chromatography and hydrophobic interaction chromatography. In one embodiment, the metal-affinity chromatography column is Ni-NTA sepharose. In a further embodiment, the hydrophobic interaction chromatography column is phenyl sepharose.

In one embodiment, the method of purifying a polypeptide comprises the steps:

(a) expressing the polypeptide in a recombinant system using an expression vector or nucleic acid sequence disclosed herein;

(b) contacting culture medium from the recombinant system comprising the polypeptide with metal-affinity beads, optionally Ni-NTA beads;

(c) eluting the bound polypeptide;

(d) contacting the eluate with hydrophobic interaction beads, optionally phenyl sepharose beads; and

(e) eluting the bound polypeptide.

In another embodiment, the method comprises the steps:

(a) expressing the polypeptide in a recombinant system using an expression vector or nucleic acid sequence disclosed herein;

(b) contacting culture medium from the recombinant system comprising the polypeptide with hydrophobic interaction beads, optionally phenyl sepharose;

(c) eluting the bound polypeptide;

(d) contacting the eluate with metal-affinity beads, optionally Ni-NTA beads; and

(e) eluting the bound polypeptide.

In a further embodiment, a cleavage recognition site is situated between the fusion tag and the polypeptide upon expression and the fusion tag can be cleaved from the polypeptide at the cleavage recognition site. In one embodiment, the fusion tag is cleaved from the polypeptide directly on the chromatography column. In another embodiment, the fusion tag is cleaved from the polypeptide off of the chromatography column.

Another aspect of the present application is a kit comprising the isolated nucleic acid disclosed herein, the expression vector disclosed herein and/or the host cell disclosed herein with an ancillary agent. For example, possible ancillary agents include buffers; stabilizers; agents that recognize the cleavage recognition site; agents to assist in the recombinant methods, including drugs or antibiotics for selectable markers; containers; vessels; and/or instructions for the use thereof.

Yet another aspect is the use of the fusion tag described herein for identifying polypeptide interactions or isolating substances that interact with a polypeptide. In one embodiment, the fusion tag may be used in pull-down experiments as shown in Example 2. For example, in one embodiment a polypeptide is cloned into a vector containing a fusion tag described herein, and the expressed polypeptide is incubated in a solution containing putative binding partners. The polypeptide may then be isolated using the fusion tag as herein described, and any substances bound to the isolated polypeptide may then be identified using techniques known to one of skill in the art.

The above disclosure generally describes the present invention. A more complete understanding can be obtained by reference to the following specific examples. These examples are described solely for the purpose of illustration and are not intended to limit the scope of the invention. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present invention:

### Examples

### Example 1

### Tandem Purification of eGFP and p53 Using a (His)6-Calmodulin (HiCaM) Fusion Tag

A major goal in the production of therapeutic proteins, subunit vaccines, as well as recombinant proteins needed for structure determination and structural proteomics is their recovery in a pure and functional state using the simplest purification procedures. Described herein is the design and use of a novel tandem (His)₆-calmodulin (HiCaM) fusion tag that combines two distinct purification strategies, namely immobilized metal-affinity (IMAC) and hydrophobic interaction chromatography (HIC), in a simple two-step procedure. Two model constructs were generated by fusing the HiCaM purification tag to the N-terminus of either the enhanced green fluorescent protein (eGFP) or the human tumor suppressor protein p53. These fusion constructs were abundantly expressed in E. coli and rapidly purified from cleared lysates by tandem IMAC/HIC to near homogeneity under native conditions. Cleavage at a thrombin recognition site between the HiCaM tag and the constructs readily produced untagged, functional versions of eGFP and human p53 that were >97% pure. The HiCaM purification strategy is rapid, makes use of widely available, high capacity, and inexpensive matrices, and therefore represents an excellent approach for large scale purification of recombinant proteins as well as small scale protein array designs.

### Materials and Methods

Cloning of constructs—To construct the tandem HiCaM fusion tag (FIG. 1), the gene coding for calmodulin was amplified from a plasmid by PCR using a forward primer that added a NcoI restriction site as well as the (His)₆ sequence (underlined) (5′-CCATGGGCAGCAGCCATCATCATCATCATCAAGCAGCGGC-3′ (SEQ ID NO:4)) to the 5′-end. A reverse primer appended a thrombin cleavage site (underlined) and NdeI restriction site (5′-AGCAGCGGCCTGGTGCCGCGCGGCAGCCATATG-3′ (SEQ ID NO:5)) at the 3′-end. The PCR product was cloned into the NcoI/NdeI sites of a pET15b vector (Novagen, San Diego, Calif.) situated upstream of the multiple cloning site, thus replacing the single (His)₆ tag of the unmodified vector. Inserts corresponding to eGFP or human p53 (1-360) were then cloned into the NdeI/BamHI sites of the modified vector by standard procedures. Plasmids were transformed into TOP10 E. coli (Invitrogen, Carlsbad, Calif.) and clones were identified by DNA sequencing.

Protein expression and purification—BL21(DE3)Star E. coli (Invitrogen) transformants harbouring each plasmid were grown in LB broth containing 100 μg/mL ampicillin. Cells were grown in shaking flasks (225 rpm) at 37° C. until they reached an OD₆₀₀ of 0.6. Protein expression was induced with 0.75 mM IPTG, and the cultures were maintained overnight with shaking at room temperature. Cell pellets from 1-L cultures (approximately 3 g) were resuspended in 30 mL of Buffer A (50 mM TrisHCl, 1 mM CaCl₂, pH 7.5) supplemented with protease inhibitors (Complete Mini EDTA-free; Roche, Mannheim, Germany) and a nonspecific nuclease (Benzonase Nuclease, 2.5 kU; Novagen). Cells were lysed by stirring with 10 g of acid-washed glass beads (Sigma, St. Louis, Mo.) as described elsewhere (Song and Jacques 1997) and cleared by centrifugation at 40,000×g for 20 min. For tandem IMAC/HIC, the cleared lysate was adjusted to 10 mM imidazole and loaded onto a 2 mL bed of Ni-NTA (Sigma-Aldrich) that had been pre-equilibrated with Buffer A. The loaded resin was washed with 10 mL of Buffer A before eluting the bound protein with 10 mL of Buffer A adjusted to 150 mM imidazole. The IMAC-purified eluate was then directly applied onto a 2 mL bed of Phenyl Sepharose 6 FF (GE Healthcare, Piscataway, N.J.) that had been pre-equilibrated with Buffer A and washed with an additional 10 mL of Buffer A. For HIC alone, the wash volume was 20 mL to match the combined wash volumes for tandem IMAC/HIC. Purified protein was recovered by elution with 5 mL of Buffer B (50 mM TrisHCl, 5 mM EGTA, pH 7.5), titrated with 50 μL of 1 M CaCl₂, and treated with 2 U/mL thrombin (GE Healthcare). Alternatively, the column was plugged and the resin was resuspended in 5 mL of Buffer B containing 2 U/mL of thrombin. The resin slurry was gently shaken overnight at room temperature. Following thrombin treatment, the untagged protein was drained from the resin and passed through a 0.5 mL bed of p-aminobenzamidine agarose (Sigma-Aldrich) to remove residual protease. Protein concentrations at various steps were measured by Coomassie Blue binding (Bio-rad), using bovine serum albumin as a standard.

Assessment of target purity—Protein samples (3 μg total protein) were analyzed by SDS-PAGE and stained with Coomassie Blue. Gel lanes were scanned by transillumination in a Bio-Rad Gel Doc XR instrument and the purity of the target protein band was quantified by densitometry using the software Quantity One (version 4.6.3, Bio-Rad).

Functional Assays of purified, untagged constructs—The excitation and emission spectra (excited at 488 nm) of eGFP were recorded from 300 to 700 nm at 25° C. in a Fluoromax-3 Spectrofluorometer. For p53(1-360), a ³²P-labelled restriction fragment harbouring an icosamer recognition sequence (5′-AGGCATGTCTAGGCATGTCT-3′ (SEQ ID NO:6)) (0.10 nM) was incubated alone, with 50 ng/μL BSA or 12 μM p53(1-360) in PBS at 25° C. with 2 μg poly(dI-dC)˜poly(dl-dC) for 1 hour before separation in a 6% polyacrylamide gel in TBM buffer at 25 V/cm. The gel was then exposed to a phosphor screen for 1 hour and imaged in a Typhoon® Phosphorimager (GE Healthcare). Human p53(1-360) (7 μM) was also crosslinked with 0.125% glutaraldehyde at 25° C. for 12 min. and separated on SDS-PAGE, followed by Coomassie Blue staining (Poon et al. 2007).

Cloning of TAP-eGFP—To construct an N-terminal TAP-tag (Protein A-TEV-CBP) version of the eGFP gene, the TAP-tag sequence was amplified from a plasmid by PCR using a forward primer that appended a NcoI restriction site (underlined) upstream of the protein A sequence (5′-CCATGGGCATGAAAGCTGATGCGCAACAA-3′ (SEQ ID NO:7)) and a reverse primer that added a NdeI restriction site (underlined) downstream of the calmodulin binding peptide sequence (5′-CATATGATCAAGTGCCCCGGAGGATGA-3′ (SEQ ID NO:8)). The PCR product was then cloned in place of the HiCaM tag into the NcoI/NdeI sites of the pET15b vector (Novagen, San Diego, Calif.) containing the eGFP gene. Correct insertion was confirmed by DNA sequencing.

Protein expression and purification of TAP-eGFP—BL21(DE3)Star E. coil (Invitrogen) transformants harbouring the plasmid were grown in LB broth containing 100 μg/mL ampicillin. Cells were grown in shaking flasks (225 rpm) at 37° C. until they reached an OD₆₀₀ of 0.6. Protein expression was induced in the presence of 0.75 mM IPTG, and the cultures were maintained overnight with shaking at room temperature. Cell pellets from 1-L cultures were resuspended in 30 mL of lysis buffer (10 mM TrisHCl, pH 8.0, 150 mM NaCl, 0.1% NP-40) supplemented with protease inhibitors (Complete Mini EDTA-free; Roche, Mannheim, Germany) and a nonspecific nuclease (Benzonase Nuclease, 2.5 kU; Novagen). The cells were lysed and cleared as described in the text, and then loaded onto a 2 mL bed of IgG sepharose (GE Healthcare, Piscataway, N.J.) that had been pre-equilibrated with lysis buffer. The loaded resin was washed with 10 mL of lysis buffer followed by 10 mL of TEV cleavage buffer (10 mM TrisHCl pH 8.0, 150 mM NaCl, 0.1% NP-40, 0.5 mM EDTA, and 1 mM DTT). The resin was re-suspended in 2 mL of TEV cleavage buffer and the bound protein was cleaved with 300 U of TEV protease (Invitrogen, Carlsbad, Calif.) overnight at 25° C. The cleaved protein was drained off the IgG column by gravity flow and supplemented with 3 volumes of CaM binding buffer (10 mM TrisHCl pH 8.0, 150 mM NaCl, 0.1% NP-40, 10 mM β-mercaptoethanol, 1 mM magnesium acetate, 1 mM imidazole, and 2 mM CaCl₂) to titrate the EDTA in the TEV cleavage buffer. The cleaved protein was loaded onto a 2 mL of CaM sepharose (GE Healthcare) pre-equilibrated in CaM binding buffer. The column was then washed with 10 ml of CaM binding buffer before eluted with 5 mL of calmodulin elution buffer (10 mM TrisHCl pH 8.0, 150 mM NaCl, 0.1% NP-40, 10 mM β-mercaptoethanol, 1 mM magnesium acetate, 1 mM imidazole, and 2 mM EGTA).

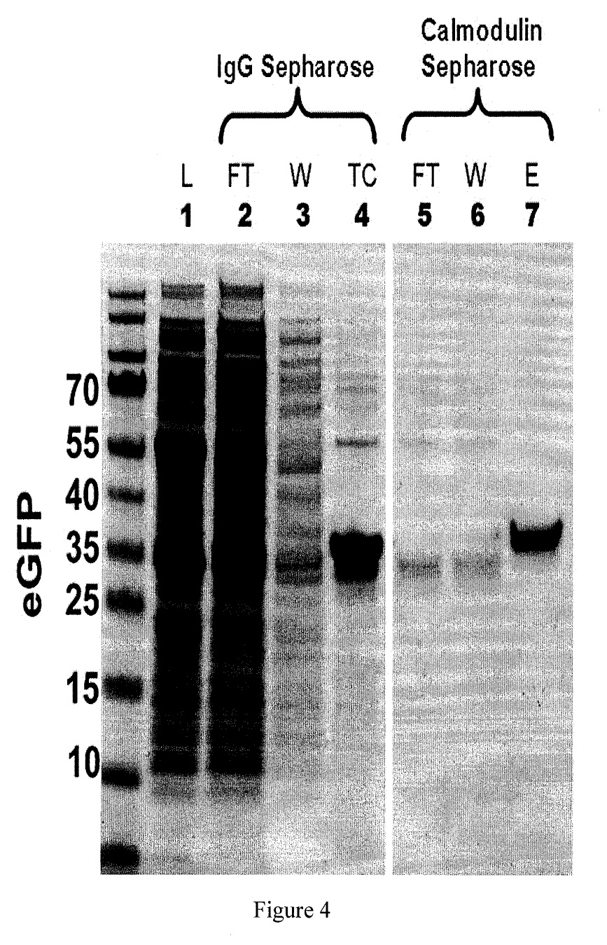

Assessment of yield and purity for the TAP tag procedure was performed by Coomassie staining and SDS-PAGE followed by densitometric analysis, respectively, as described for HiCaM-tagged eGFP (FIG. 4 and Table 2). Values shown in Table 2 are averages (±standard error) for triplicate experiments. Note that the final recovered construct was CBP-eGFP. Thus, the TAP tag procedure produced CBP-eGFP at a purity and efficiency of (94±2)% and (24±9)%, respectively. By comparison, tandem IMAC/HIC (i.e., the HiCaM procedure) produced untagged eGFP at a purity and efficiency of (98±1)% and (48±11)%, respectively (Table 1). A cost comparison between the two purification schemes is also provided in Table 3.

### Results

Expression and purification of HiCaM-tagged eGFP and p53(1-360) by tandem IMAC/HIC—A novel and universal dual affinity purification tag, HiCaM tag, was designed to address the need for rapid and effective purification of recombinant proteins to homogeneity under native conditions. Specifically, the HiCaM-tag (FIG. 1) was fused to the N-terminus of either eGFP or p53(1-360) via a thrombin cleavage site and the resulting protein constructs were expressed in E. coli. Both fusion proteins were well expressed to over 10 mg/L culture (Table 1) indicating that the addition of the HiCaM tag did not adversely affect their expression.

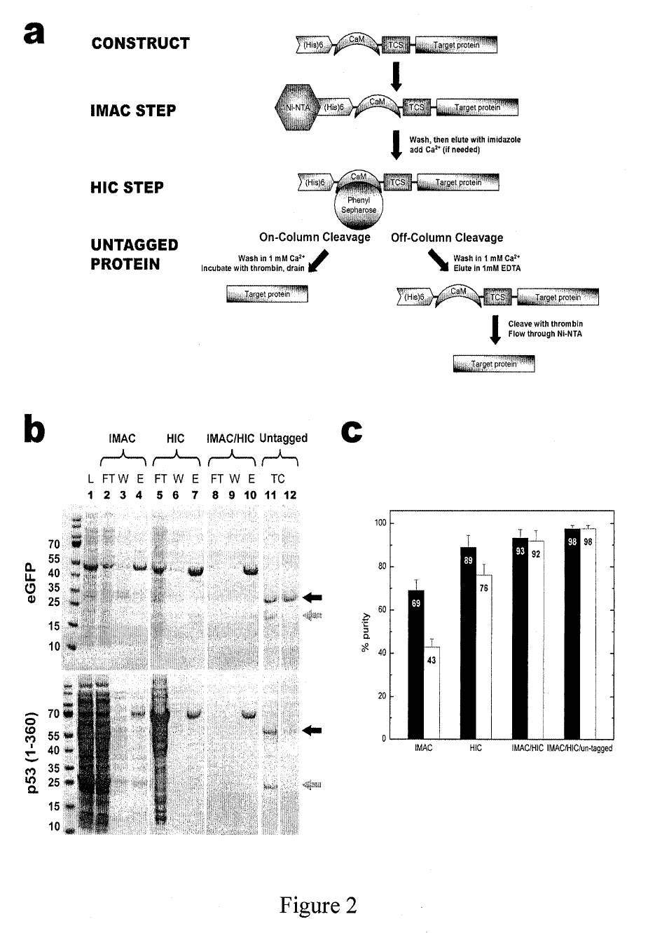

HiCaM-tagged protein samples were then purified from cleared lysates by IMAC on Ni-NTA sepharose and followed immediately by HIC on phenyl sepharose (FIG. 2A). Since the buffers for IMAC and HIC are compatible, buffer exchange is unnecessary and the IMAC eluate can be applied directly onto phenyl sepharose. SDS-PAGE analysis of fractions collected at various steps showed contaminating protein bands in the eluates from IMAC or HIC alone (FIG. 2B: Lanes 4 and 7), whereas coupled IMAC/HIC purification profiles appeared homogeneous (FIG. 2B: Lanes 10 to 12). Purified, untagged proteins were recovered, in separate experiments, in an off-column procedure or directly on-column by treatment of thrombin. In the off-column method, tagged proteins were eluted from phenyl sepharose in the presence of EGTA which was then titrated with excess Ca²⁺ before addition of thrombin. For on-column cleavage, washed resin was resuspended and treated with thrombin to release untagged proteins in the flow-through. Densitometric analyses (FIG. 2C) indicated that products from both methods were essentially identical in purity (>97%), although on-column cleavage represents a simpler approach at a small cost in terms of yield (Table 1). Final recovery of purified eGFP and p53(1-360) was in excess of 15 (32%) and 1.3 mg (12%) per L culture, respectively, based on 2 mL each of Ni-NTA and phenyl sepharose. Since significant amounts of target constructs appeared in the column flow-through (FIG. 2B), one skilled in the art would appreciate that an appropriate scale-up would further increase the yield.

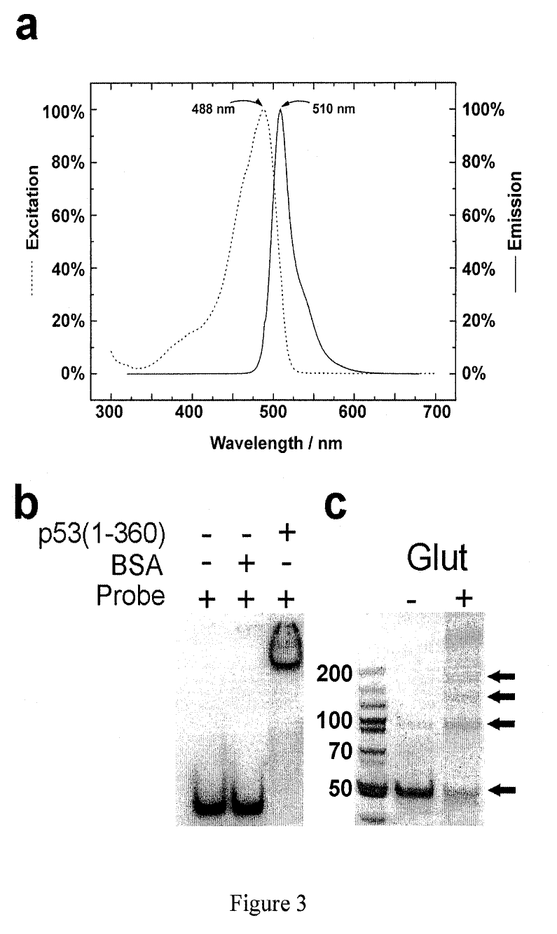

To verify that the tandem IMAC/HIC purification of HiCaM-tagged constructs under native conditions produced functional proteins, the excitation and emission spectra of the purified, untagged eGFP were recorded and confirmed the recovery of a fluorescent protein (FIG. 3A) (Shaner et al. 2005). Also, purified, untagged human p53(1-360) was shown to bind to a recognition DNA sequence by gel mobility shift (FIG. 3B). Finally, SDS-PAGE analysis after glutaraldehyde crosslinking of p53(1-360) revealed a ladder of bands characteristic tetramers formed in solution via its tetramerization domain (residues 325 to 355) (FIG. 3C) (Lee et al. 1994; Poon et al. 2007). Therefore, the native conformation of both proteins was not compromised by their expression as fusion proteins with an N-terminal HiCaM-tag.

Comparison with another dual-tag purification strategy—Although tandem IMAC/HIC purification of HiCaM-tagged eGFP and p53(1-360) produced essentially homogeneous preparations, we also purified, for comparison, eGFP tagged with an N-terminal “TAP tag”, which consists of two IgG binding domains of ProtA and CBP separated by a cleavage site for the TEV protease (Rigaut et al. 1999; Puig et al. 2001). The TAP tag is widely used to purify protein-protein complexes in proteomic analysis. Briefly, the TAP tagged construct was purified on IgG-sepharose (which interacts with ProtA), released by TEV cleavage, followed by a second purification on CaM-sepharose (which binds CBP in the presence of Ca²⁺). The procedure was adjusted to use the same volumes of the two resins and their respective washing buffers as the HiCaM procedure described above. Under these conditions, TAP tag purification produced eGFP at a purity and efficiency (fraction of purified target recovered) of (94±2)% and (24±9)% (FIG. 4), respectively, compared with (98±1)% and (48±11)% for the HiCaM procedure (Table 1). Thus, for the model protein eGFP, both schemes resulted in similarly pure proteins but the tandem IMAC/HIC gave a two-fold increase in efficiency. In addition, the TAP tag procedure required a buffer change after TEV cleavage for binding onto CaM sepharose and the final purified TAP-tagged product was CBP-eGFP, not untagged eGFP. The presence of this 28-amino acid tag, however, did not qualitatively affect the fluorescent properties of the eGFP protein. In addition, the cost of the TAP tag purification approach (for the resins and TEV protease) was significantly higher than the HiCaM procedure (Table 3).

A new method for purifying recombinant proteins was developed by fusing a (His)₆-CaM (HiCaM) tag at the N-terminus of target proteins (FIG. 1). We have purified, as examples, eGFP and p53(1-360) under native conditions to apparent homogeneity (as judged by densitometric analysis of Coomassie-stained SDS-PAGE gels) by a tandem purification strategy that combines IMAC and HIC in a simple procedure (FIG. 2A). The level of purity observed after tandem IMAC/HIC was not reproduced by either IMAC or HIC alone, even after adjustment of wash volumes (FIGS. 2B and 2C). Moreover, the recovered target proteins were functional after removing the HiCaM tag (native fluoresence for eGFP; specific DNA-binding and tetramerization for p53) (FIG. 3), indicating that the HiCaM tag, once removed, did not perturb the native conformation of the target proteins.

Features of tandem IMAC/HIC and the (His)₆-CaM tag—The contiguous combination of (His)₆ and CaM results in a convenient, relatively small (19 kDa) purification tag for tandem IMAC/HIC purification. Though larger than peptide-based affinity tags, the HiCaM-tag is one of the smallest protein-based purification tags known: smaller than MBP (40 kDa), protein G (27 kDa), the popular GST tag (28 kDa), and is only marginally larger than ProtA itself (15 kDa). It makes use of two widely available, high-capacity, and inexpensive matrices (Ni- or Co-NTA and phenyl sepharose). CaM is a highly soluble protein regardless of its Ca²⁺ load, and did not (as indicated by our soluble yield of tagged eGFP and p53) adversely affect the expression of the two fusion constructs (>10 mg/L culture; Table 1). In fact, a structural homolog of CaM, the calcium-binding protein of Entamoeba histolytica, has been used as a solubilizing fusion partner for a recombinant multiple-epitope polypeptide (Reddi et al. 2002).

The use of CaM alone as a fusion tag is known in the art (see for example Neri et al. 1995; Schauer-Vukasinovic and Daunert 1999; Vaillancourt et al. 2000; Schauer-Vukasinovic et al. 2002; Melkko and Neri 2003). Since various high-affinity CaM ligands are known (e.g., CBPs, phenothiazines), a person skilled in the art will appreciate that other CaM-specific strategies are possible in addition to HIC when using the HiCaM fusion tag described herein. For example, CaM-tagged GFP has been purified on phenothiazine-silica prepared by Daunert and coworkers (Schauer-Vukasinovic and Daunert 1999; Schauer-Vukasinovic et al. 2002). To our knowledge, affinity matrices harbouring specific ligands for CaM are not available commercially. Accordingly, a HIC matrix such as phenyl sepharose is a preferred embodiment given its robustness, high capacity, wide availability, and very low cost.

Since the buffers for Ni-NTA and phenyl sepharose are compatible, the entire IMAC/HIC procedure can be rapidly performed in a pour-on column format with no delay between purification and minimal optimization of wash and elution conditions for either stage (FIG. 2A). The tight retention and high capacity of the IMAC and HIC matrices for (His)₆ and Ca²⁺-CaM, respectively, permit stringent and extensive washing of the resins. In addition, the disparate nature of the interactions in the IMAC and HIC matrices makes it very unlikely that a host contaminant will have the appropriate physicochemical characteristics for strong interactions with both matrices and sensitivity to imidazole and Ca²⁺.

Conclusion—Tandem IMAC/HIC purification with the HiCaM tag provides a novel purification method that offers a rapid and easy way to recover pure proteins at low cost. Two target proteins, in this case eGFP and human p53(1-360), were produced in high yield showing that this process is scalable while maintaining their functional (native) conformations. Although we have engineered the HiCaM tag in a pET15b vector, it is easily adapted for cloning into any other expression vector (bacterial, yeast, or mammalian) or baculovirus transfer vector (for expression in insect cells). Overall, the tandem IMAC/HIC strategy allows for the purification of recombinant proteins with little-to-no optimization and advances the field of protein purification in the aspect of achieving greater purity using a simple, two-step procedure.

### Example 2

### Use of HiCam Purification Tags in Pull-Down Experiments with Shiga-Like Toxin 1

In one embodiment, the fusion tag described herein may be used in pull down experiments to investigate protein interactions. In the course of investigations into the binding of the catalytic subunit of shiga-like toxin 1 (SLT-1) to ribosomal proteins P0, P1 and P2, in vitro pull-down experiments using fusion protein-tagged C-terminal peptides corresponding to the common 7, 11, and 17 terminal residues of ribosomal proteins P1 and P2 confirmed that the A1 chain of SLT-1 as well as the A chain of ricin bind to this shared C-terminal peptide motif.

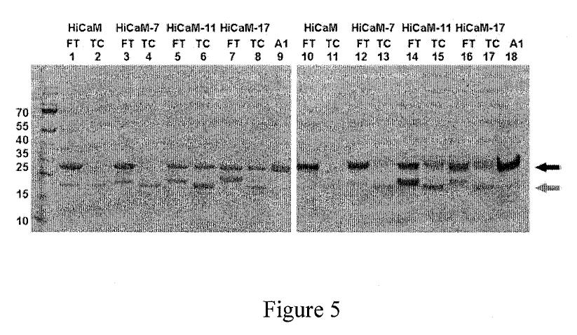

Various lengths of this peptide motif were fused to the C terminus of calmodulin as part of the HiCaM tandem purification tag described herein. The resulting fusion constructs were incubated with 8 μM SLT-1 A1 chain or Ricin A Chain (RTA) (FIG. 5, lanes 9 and 18, respectively) in an effort to delimit a minimum binding motif. Toxins that specifically bound these HiCaM-tagged peptides were pulled down on phenyl sepharose and subsequently released by treatment with thrombin (FIG. 5 lanes labeled TC). The results show that the SLT-1 A1 chain as well as RTA were recovered after thrombin cleavage only in the presence of HiCaM peptides corresponding to the C-terminal 11 or 17, but not to the last seven amino acids or to the HiCaM-tag alone (FIG. 5, black arrow).

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### REFERENCE LIST

Coulombe, P., and Meloche, S. 2002. Dual-tag prokaryotic vectors for enhanced expression of full-length recombinant proteins. Anal Biochem 310: 219-222.
Donnelly, M. I., Zhou, M., Millard, C. S., Clancy, S., Stols, L., Eschenfeldt, W. H., Collart, F. R., and Joachimiak, A. 2006. An expression vector tailored for large-scale, high-throughput purification of recombinant proteins. Protein Expr Purif 47: 446-454.
Goda, N., Tenno, T., Inomata, K., Iwaya, N., Sasaki, Y., Shirakawa, M., and Hiroaki, H. 2007. LBT/PTD dual tagged vector for purification, cellular protein delivery and visualization in living cells. Biochim Biophys Acta 1773: 141-146.
Gopalakrishna, R., and Anderson, W. B. 1982. Ca²⁺-induced hydrophobic site on calmodulin: application for purification of calmodulin by phenyl-Sepharose affinity chromatography. Biochem. Biophys. Res. Commun. 104: 830-836.
Honey, S., Schneider, B. L., Schieltz, D. M., Yates, J. R., and Futcher, B. 2001. A novel multiple affinity purification tag and its use in identification of proteins associated with a cyclin-CDK complex. Nucleic Acids Res 29: E24.
Kapust, R. B., and Waugh, D. S. 1999. Escherichia coli maltose-binding protein is uncommonly effective at promoting the solubility of polypeptides to which it is fused. Protein Sci 8: 1668-1674.
Lee, W., Harvey, T. S., Yin, Y., Yau, P., Litchfield, D., and Arrowsmith, C. H. 1994. Solution structure of the tetrameric minimum transforming domain of p53. Nat Struct Biol 1: 877-890.
Lichty, J. J., Malecki, J. L., Agnew, H. D., Michelson-Horowitz, D. J., and Tan, S. 2005. Comparison of affinity tags for protein purification. Protein Expr Purif 41: 98-105.
McLure, K. G., and Lee, P. W. 1998. How p53 binds DNA as a tetramer. Embo J 17: 3342-3350.
Melkko, S., and Neri, D. 2003. Calmodulin as an affinity purification tag. Methods Mol. Biol. 205: 69-77.
Mueller, U., Bussow, K., Diehl, A., Bartl, F. J., Niesen, F. H., Nyarsik, L., and Heinemann, U. 2003. Rapid purification and crystal structure analysis of a small protein carrying two terminal affinity tags. J Struct Funct Genomics 4: 217-225.
Neri, D., de Lalla, C., Petrul, H., Neri, P., and Winter, G. 1995. Calmodulin as a versatile tag for antibody fragments. Biotechnology (NY) 13: 373-377.
Panagiotidis, C. A., and Silverstein, S. J. 1995. pALEX, a dual-tag prokaryotic expression vector for the purification of full-length proteins. Gene 164: 45-47.
Pathak, R., and Imperiali, B. 1997. A dual affinity tag on the 64-kDa NIt1p subunit allows the rapid characterization of mutant yeast oligosaccharyl transferase complexes. Arch Biochem Biophys 338: 1-6.
Pavlou, A. K., and Reichert, J. M. 2004. Recombinant protein therapeutics—success rates, market trends and values to 2010. Nat Biotechnol 22: 1513-1519.
Poon, G. M., Brokx, R. D., Sung, M., and Gariepy, J. 2007. Tandem dimerization of the human p53 tetramerization domain stabilizes a primary dimer intermediate and dramatically enhances its oligomeric stability. J Mol Biol 365: 1217-1231.
Puig, O., Caspary, F., Rigaut, G., Rutz, B., Bouveret, E., Bragado-Nilsson, E., Wilm, M., and Seraphin, B. 2001. The Tandem Affinity Purification (TAP) Method: A General Procedure of Protein Complex Purification. Methods 24: 218-229.
Reddi, H., Bhattacharya, A., and Kumar, V. 2002. The calcium-binding protein of Entamoeba histolytica as a fusion partner for expression of peptides in Escherichia coli. Biotechnol Appl Biochem 36: 213-218.
Rigaut, G., Shevchenko, A., Rutz, B., Wilm, M., Mann, M., and Séraphin, B. 1999. A generic protein purification method for protein complex characterization and proteome exploration. Nat. Biotechnol. 17: 1030-1032.
Sachdev, D., and Chirgwin, J. M. 1998. Solubility of proteins isolated from inclusion bodies is enhanced by fusion to maltose-binding protein or thioredoxin. Protein Expr Purif 12: 122-132.
Schauer-Vukasinovic, V., and Daunert, S. 1999. Purification of recombinant proteins based on the interaction between a phenothiazine-derivatized column and a calmodulin fusion tail. Biotechnol. Prog. 15: 513-516.
Schauer-Vukasinovic, V., Deo, S. K., and Daunert, S. 2002. Purification method for recombinant proteins based on a fusion between the target protein and the C-terminus of calmodulin. Anal. Bioanal. Chem. 373: 501-507.
Shaner, N. C., Steinbach, P. A., and Tsien, R. Y. 2005. A guide to choosing fluorescent proteins. Nat. Methods 2: 905-909.
Song, D. D., and Jacques, N. A. 1997. Cell disruption of Escherichia coli by glass bead stirring for the recovery of recombinant proteins. Anal. Biochem. 248: 300-301.
Stevens, R. C. 2000. Design of high-throughput methods of protein production for structural biology. Structure 8: R177-185.
Tropea, J. E., Cherry, S., Nallamsetty, S., Bignon, C., and Waugh, D. S. 2007. A generic method for the production of recombinant proteins in Escherichia coli using a dual hexahistidine-maltose-binding protein affinity tag. Methods Mol Biol 363: 1-19.
Vaillancourt, P., Zheng, C. F., Hoang, D. Q., and Breister, L. 2000. Affinity purification of recombinant proteins fused to calmodulin or to calmodulin-binding peptides. Methods Enzymol. 326: 340-362.
Walsh, G. 2005. Biopharmaceuticals: recent approvals and likely directions. Trends Biotechnol 23: 553-558.
Winkler, G. S., Vermeulen, W., Coin, F., Egly, J. M., Hoeijmakers, J. H., and Weeda, G. 1998. Affinity purification of human DNA repair/transcription factor TFIIH using epitope-tagged xeroderma pigmentosum B protein. J Biol Chem 273: 1092-1098.
Yeliseev, A., Zoubak, L., and Gawrisch, K. 2007. Use of dual affinity tags for expression and purification of functional peripheral cannabinoid receptor. Protein Expr Purif 53: 153-163.

TABLE 1
Summary of tandem IMAC/HIC purification of HiCaM-tagged
recombinant constructs (per L culture)^{a}

| | Total protein/ | | Yield^{c}/ | |
|---|---|---|---|---|
| | mg | Purity^{b}/% | mg | Efficiency^{d}/% |
| | eGFP | | | |
| Cleared lysate | 203 ± 14 | 23 ± 4 | 47 ± 9 | |
| IMAC eluate | 42 ± 3 | 69 ± 5 | 29 ± 3 | 62 ± 14 |
| HIC eluate | 27 ± 3 | 93 ± 4 | 25 ± 3 | 54 ± 12 |
| HIC/on-column | 23 ± 3 | 98 ± 1 | 23 ± 3^{e} | 48 ± 11^{e} |
| cleavage | | | | |
| | p53(1-360) | | | |
| Cleared lysate | 220 ± 15 | 5 ± 3 | 11 ± 7 | |
| IMAC eluate | 5.5 ± 1.0 | 43 ± 5 | 2.4 ± 0.5 | 22 ± 14 |
| HIC eluate | 2.5 ± 0.7 | 92 ± 5 | 2.3 ± 0.7 | 21 ± 14 |
| HIC/on-column | 1.3 ± 0.3 | 98 ± 1 | 1.3 ± 0.3^{e} | 12 ± 7^{e} |
| cleavage | | | | |

^{a}Values are averages of experiments performed in triplicate ± standard error.
^{b}Determined by densitometric analysis of Coomassie-stained SDS-PAGE.
^{c}Yield = total protein × purity.
^{d}Efficiency is calculated as the residual yield at each step relative to the cleared lysate.
^{e}Values represent purified, untagged proteins.

TABLE 2
Summary of IgG/CaM affinity purification of TAP-eGFP

| | eGFP | | | |
|---|---|---|---|---|
| | Total | | | |
| | protein/ | Purity/ | Yield^{a}/ | |
| | mg | % | mg | Efficiency^{b}/% |
| Cleared lysate | 242 ± 17 | 15 ± 3 | 36 ± 8 | |
| IgG chromatography/ | 18 ± 4 | 58 ± 5 | 10 ± 2 | 29 ± 9 |
| TEV cleavage | | | | |
| CaM affinity | 9.3 ± 2.8 | 94 ± 2 | 8.7 ± 2.6^{c} | 24 ± 9^{c} |
| chromatography | | | | |

^{a}Yield = total protein × purity.
^{b}Efficiency is calculated as the residual yield at each step relative to the cleared lysate.
^{c}Values represent CBP-eGFP.

TABLE 3
Comparison of chromatographic matrices for purification of
HiCaM ((His)₆-CaM) and TAP (Protein A-CBP) fusion tags^{a}

| | | Nominal | Ligand | | |
|---|---|---|---|---|---|
| Fusion | Immob. | capacity | density | Price | |
| tag | ligand | mg/mL | μmol/mL | USD/mL resin^{b} | |
| Oligo-His | Ni²⁺ | >15^{c} | N/A | $5.87^{d} | HIS-Select Ni |
| | | | | | Affinity Gel |
| CaM | Phenyl | N/A | 40 | $1.35^{e} | Phenyl |
| | | | | | Sepharose 6 FF |
| Protein A | IgG | >2 | N/A | $29.70^{e} | IgG Sepharose |
| | | | | | FF |
| CBP | CaM | N/A | <0.076 | $30.00^{e} | Calmodulin |
| | | | | | sepharose 4B |

^{a}Data: GE Healthcare and Sigma-Aldrich websites
^{b}Normalized price for the largest available pack size
^{c}For a 30 kDa His-tagged protein
^{d}Current list price, Sigma-Aldrich Chemicals
^{e}Current list price, GE Healthcare

1. An isolated nucleic acid sequence comprising a nucleic acid sequence that encodes a fusion tag, wherein the fusion tag comprises an affinity tag, optionally a polyhistidine tag such as (His)₆, attached to a polypeptide comprising one or more EF hand motif(s), optionally calmodulin or a fragment thereof.

2. The isolated nucleic acid of claim 1, wherein the polypeptide comprising one or more EF hand motif(s) comprises one or more of the EF-hand motifs of calmodulin, and wherein the affinity tag is attached via a fusion linker to the polypeptide comprising one or more EF hand motif(s).

3. (canceled)

4. The isolated nucleic acid sequence of claim 2, wherein the fusion linker is 1 to 100 amino acids.

5. The isolated nucleic acid sequence of claim 1, wherein the affinity tag is N-terminus to the polypeptide comprising one or more EF hand motif(s).

6. The isolated nucleic acid sequence of claim 1, wherein the affinity tag is C-terminus to the polypeptide comprising one or more EF hand motif(s).

7. The isolated nucleic acid sequence of claim 1, wherein the isolated nucleic acid sequence further encodes a polypeptide, wherein the polypeptide is attached to the fusion tag upon expression, and wherein the polypeptide is attached via a linker to the fusion tag.

8. (canceled)

9. The isolated nucleic acid sequence of claim 7, wherein the linker comprises a cleavage recognition site.

10. The isolated nucleic acid sequence of claim 9, wherein the cleavage recognition site is a thrombin cleavage recognition site.

11. The isolated nucleic acid sequence of claim 7, wherein the polypeptide is N-terminus to the fusion tag.

12. The isolated nucleic acid sequence of claim 7, wherein the polypeptide is C-terminus to the fusion tag.

13. The isolated nucleic acid sequence of claim 1, wherein the isolated nucleic acid sequence further encodes a polypeptide and wherein the polypeptide is attached between the affinity tag and polypeptide comprising one or more EF hand motif(s) upon expression.

14. The isolated nucleic acid sequence of claim 1, wherein the isolated nucleic acid sequence further encodes a cleavage recognition site and wherein the cleavage recognition site is attached to the fusion tag upon expression, and wherein the cleavage recognition site is attached via a linker to the fusion tag.

15. (canceled)

16. (canceled)

17. The isolated nucleic acid sequence of claims 14, wherein the cleavage recognition site is N-terminus to the fusion tag.

18. The isolated nucleic acid sequence of claim 14, wherein the cleavage recognition site is C-terminus to the fusion tag.

19. The isolated nucleic acid sequence of claim 14, wherein the cleavage recognition site is a thrombin cleavage recognition site.

20. An isolated nucleic acid sequence comprising the nucleic acid sequence of SEQ ID NO:3.

21. An expression vector comprising the nucleic acid sequence of claim 20.

22. A host cell comprising the expression vector of claim 21.

23. An isolated protein encoded by the nucleic acid sequence of claim 20.

24. A method of purifying a polypeptide comprising:
(a) expressing the polypeptide using an expression vector comprising the nucleic acid sequence of claim 6; and
(b) purifying the polypeptide using two different purification methods, immobilized metal-affinity chromatography and hydrophobic interaction chromatography.

25.-30. (canceled)


SEQUENCE LISTING (8 sequences)

SEQ ID NO: 1 - protein, 152 aa, Homo sapiens
MADQLTEEQIAEFKEAFSLFDKDGDGTITTKELGTVMRSLGQNPTEAELQDMINEVDADDLPGNGTIDFPEFLTMMARKMKDTDSEEEIREAFRVFDKDGNGYISAAELRHVMTNLGEKLTDEEVDEMIREADIDGDGQVNYEEFVQMMTAK

SEQ ID NO: 2 - protein, 173 aa, Artificial Sequence
  Fusion tag
MGSSHHHHHHSSGMADQLTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAKSSGLVPRGSHM

SEQ ID NO: 3 - DNA, 521 bp, Artificial Sequence
  Fusion tag
ccatgggcagcagccatcatcatcatcatcacagcagcggcatggctgatcagctgaccgaagaacagattgctgaattcaaggaagccttctccctatttgataaagatggcgatggcaccatcacaacaaaggaacttggaactgtcatgaggtcactgggtcagaacccaacagaagctgaattgcaggatatgatcaatgaagtggatgctgatggtaatggcaccattgacttccccgaatttttgactatgatggctagaaaaatgaaagatacagatagtgaagaagaaatccgtgaggcattccgagtctttgacaaggatggcaatggttatatcagtgcagcagaactacgtcacgtcatgacaaacttaggagaaaaactaacagatgaagaagtagatgaaatgatcagagaagcagatattgatggagacggacaagtcaactatgaagaattcgtacagatgatgactgcaaaaagcagcggcctggtgccgcgcggcagccatatg

SEQ ID NO: 4 - DNA, 41 bp, Artificial Sequence
  Primer
ccatgggcagcagccatcatcatcatcatcacagcagcggc

SEQ ID NO: 5 - DNA, 33 bp, Artificial Sequence
  Primer
agcagcggcctggtgccgcgcggcagccatatg

SEQ ID NO: 6 - DNA, 20 bp, Artificial Sequence
  Restriction fragment harbouring an icosamer recognition sequence
aggcatgtctaggcatgtct

SEQ ID NO: 7 - DNA, 29 bp, Artificial Sequence
  Primer
ccatgggcatgaaagctgatgcgcaacaa

SEQ ID NO: 8 - DNA, 27 bp, Artificial Sequence
  Primer
catatgatcaagtgccccggaggatga